(19) United States
(12) Patent Application Publication
(10) Pub. No.: US 2007/0276123 A1
(43) Pub. Date: Nov. 29, 2007

(54) Amyloid Formation

(76) Inventors: Nigel Grant Larsen, Christchurch (NZ); Kevin H. Sutton, Christchurch (NZ); Juliet A. Gerrard, Christchurch (NZ); Sarah H. Waterhouse, Christchurch (NZ)

Correspondence Address: EDWARDS ANGELL PALMER & DODGE LLP, P.O. BOX 55874, BOSTON, MA, 02205, US

(21) Appl. No.: 10/574,877

(22) PCT Filed: Oct. 11, 2004
(86) PCT No.: PCT/NZ04/00247

§ 371 Date: May 31, 2007

(30) Foreign Application Priority Data
Oct. 9, 2003 (NZ) NZ 528780

(51) Int. Cl. C07K 14/415; C07K 1/00; C07K 14/62

(52) U.S. Cl. 530/303; 530/350; 530/375

(57) ABSTRACT

A protein must fold into a specific conformation in order to carry out its intended function. Failure to fold correctly may lead to self-association and aggregation, and formation of a highly ordered and insoluble form of protein—the amyloid fibril. Amyloid fibrils are associated with a growing number of human diseases and are thus of significant medical interest. Current interest in amyloid has also grown within the wider protein science research community, based on the discovery that these unusual structures are likely to represent a generic form of all proteins. The invention provides amyloid fibrils, for novel biomaterials development, from a heterogeneous protein mixture. In particular it provides a protein derived as a secondary product from an industrial production process (wheat gluten) to make amyloid fibrils for novel biomaterials.

### FIELD OF THE INVENTION

The invention relates to the formation of amyloid, particularly to the formation of amyloid from wheat flour proteins.

### BACKGROUND OF THE INVENTION

Amyloid is the term used to describe a highly ordered and insoluble type of protein that forms as the result of a normally soluble protein aggregating via a self-association process. In the body, this process is believed to be linked to a breakdown in the systems that ensure efficient protein synthesis and folding, and the formation of amyloid is associated with a number of human diseases, including Alzheimer's, Parkinson's, and the transmissible spongiform encephalopathies (Dobson, 2001). Of further interest, is the organised and repetitive structure of the amyloid fibril that can also be considered a form of natural scaffolding, with many surface residues on to which functional groups may be incorporated. It is possible that this feature may be exploited to develop novel biomaterials.

How to form amyloid fibrils is well established for many proteins, including the SH3 domain from bovine phosphatidyl-inositol-3′-kinase (Guijarro, 1998), the small α/β protein acylphosphatase (Chiti, 1999) and proteins associated with human disease, including transthyretin, lysozyme (Bellottí, 1999), and insulin (Nielsen, 2001; Maloy, 1981). Methods include the introduction of heat and/or acid and/or protein denaturants. The vast majority of literature available on the formation of amyloid describes the behaviour of pure proteins of low molecular weight, or protein fragments. These model and synthetic peptide species are proving to be useful systems for the controlled analysis of amyloid assembly and structure (Zanuy, 2003; Takahashi, 1998). However, a reliance on these forms of protein could potentially represent a major obstacle for industrial production, as sourcing large volumes of pure protein of the required size may prove to be costly and hence a nonviable option. An economical alternative would be to source heterogeneous mixtures of protein that were able to form amyloid fibrils, although this has not yet been achieved in vitro.

The protein fractions extracted from wheat flour are rich in glutamine residues and polyglutamine (polyQ) repeats, which are linked with the formation of amyloid-like structure (Chen, 2002). Wheat flour is inexpensive, easily accessible, and can be separated into characterised protein fractions. These features mean that even though wheat flour proteins are far larger than most of the proteins that have so far been reported to form amyloid (and, indeed, may be considered some of the largest proteins that exist, with relative sizes ranging into the tens of millions of Daltons (Wrigley 1996)), they fit many of the criteria required of a protein for use on an industrial scale. The potential to develop these plant-derived proteins into biomaterials holds great appeal for a number of reasons. Firstly, wheat is an abundant and non-toxic source of protein. Wheat protein (as gluten) is currently produced on a large scale as a secondary product during the manufacture of starch, meaning it can be sourced relatively cheaply. Secondly, specific fractions can be produced on a large scale using bacterial expression systems, and with a high level of purity (Dowd and Bekes 2002). Thirdly, it has already been demonstrated that the gluten component of wheat protein can form extensive matrix networks (Graveland et al. 1995), involving both covalent and non-covalent interactions.

### OBJECT OF THE INVENTION

It is an object of the invention to produce amyloid fibrils from a readily available protein source, and/or to provide a procedure that is able to produce amyloid fibrils on an industrial scale, or to at least provide the public with a useful choice.

### SUMMARY OF THE INVENTION

The invention provides a method of amyloid fibril formation using a heterogeneous source of protein starting material.

Preferably a source of protein possessing a high glutamine content is used as the starting material.

More especially the starting protein is from a wheat source, or a wheat protein sequence from a microbiological expression system.

The invention also provides a method of producing amyloid fibrils from a high molecular weight, heterogeneous source of starting protein material. The starting material may be wheat protein:

In particular, the invention provides a method of producing amyloid fibrils derived from wheat, comprising:
a) Providing wheat protein, crudely fractionated from a milled flour
b) Separating a heterogeneous protein mixture on the basis of solubility
c) Obtaining protein solutions containing a broad range of proteins of varying molecular weights and compositions
d) Incubation of these fractions at moderate temperatures, typically in the presence of specific compounds known to destabilise a protein's structure, to induce the formation of amyloid

Thus the invention provides the production of amyloid from a heterogeneous protein system in vitro.

The addition of denaturing compounds, such as urea (see Table 3), and incubation of the extracted wheat flour proteins at 25° C. or 37° C., for a period of several weeks, is sufficient to induce the formation of amyloid-like structures.

A denaturing compound may be added to induce the formation of amyloid-like structures. The denaturing compound may be one or more of urea, a thiol containing reductant (e.g. dithiothreitol (DTT)) oran acid (e.g. H₂SO₄, HCl).

The denaturing compound is preferably added with the protein source at 25° C. or 37° C. or 50° C. for up to 105 days.

An extraneous amyloid fibril (eg insulin fibril) may be added as a “seed” to induce amyloid fibril formation in the wheat protein treatments.

The invention also provides amyloid fibrils when produced by any of the above methods.

### DESCRIPTION OF THE FIGURES

The invention will now be described, by way of example only and with reference to the following figures in which:

FIG. 1 shows detail relating to the yields of each extracted protein fraction, using the procedure described in section 2. In particular, it shows approximate protein concentrations of the major protein groups extracted from wheat flour prior to lyophilisation.

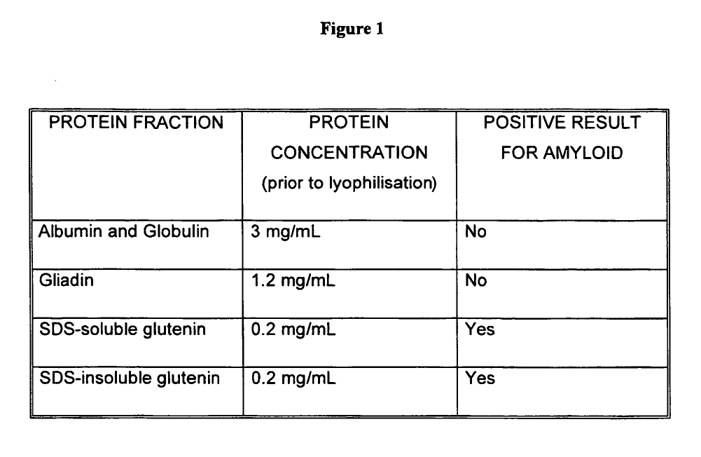

FIG. 2a shows a representative positive result for the presence of amyloid, as determined by the ThT fluorescence assay. This result was obtained for a 10 mg/ml solution of SDS-insoluble glutenin protein incubated in modified phosphate buffer (0.05M, 0.5% SDS (w/v), 0.5 M HCl) at 25° C. for up to 65 days. Average fluorescence emission intensity at 482 nm (λEx 450 nm) was measured for dilute protein solutions containing ThT (5 μM) in Tris buffer (50 mM, 100 mM NaCl), pH 7.5. Samples analysed in duplicate, and curve fitted according to the method of Nielsen (2001). Error bars represent the standard error of the mean. a.u., arbitary units.

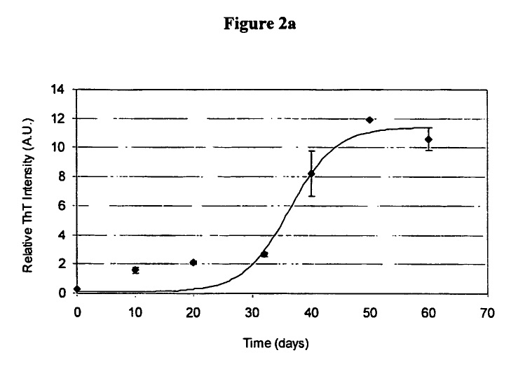

FIG. 2b shows a ThT assay result for a 10 mg/ml solution of SDS-soluble glutenin protein incubated in modified phosphate buffer (0.05M, 0.5% SDS (w/v), 0.5 M H₂SO₄) at 25° C. for up to 105 days. Average fluorescence emission intensity at 482 nm QEx 450 nm) was measured for dilute protein solutions containing ThT (5 μM) in Tris buffer (50 mM, 100 mM NaCl), pH 7.5. Samples analysed in triplicate. a.u., arbitary units.

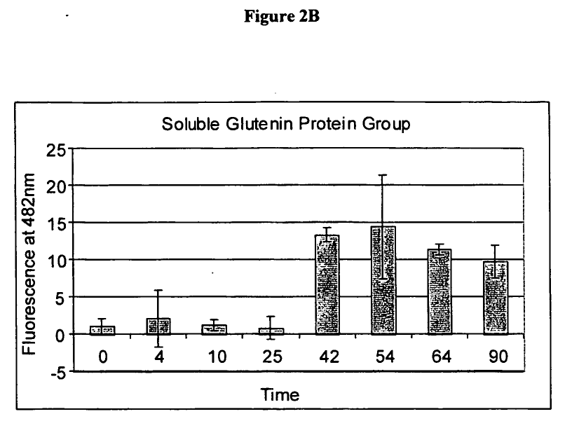

FIG. 3 shows a representative positive result for the presence of amyloid, as visualized using the imaging technique of transmission electron microscopy (TEM). These results were obtained for a 10 mg/ml solution of SDS-insoluble glutenin protein incubated in phosphate buffer (0.05M, 0.5% SDS (w/v)) at 25° C. for up to 105 days. A) SDS-insoluble glutenin samples incubated for 54 days. B) C) D) SDS-insoluble glutenin samples incubated undisturbed for 105 days. Protein samples were diluted to 1 mg/ml before analysis. All scale bars represent 200 nm.

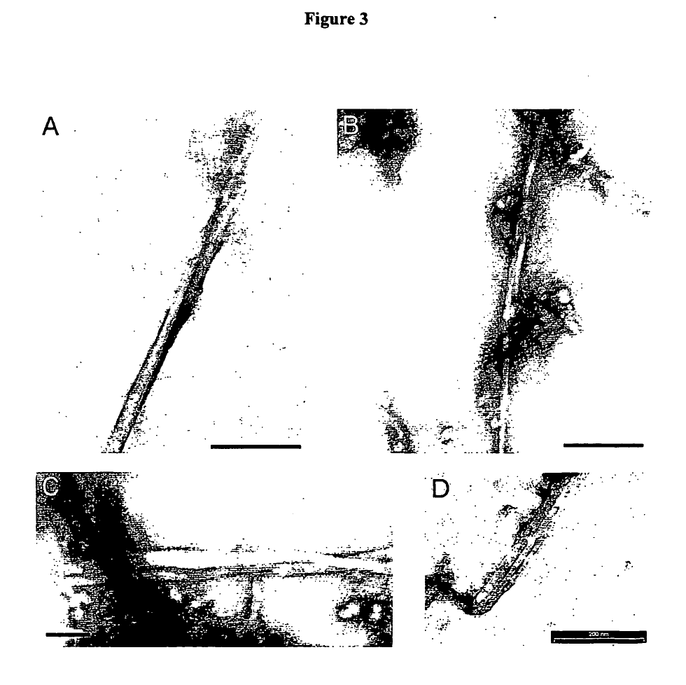

### DETAILED DESCRIPTION OF THE INVENTION

The inventors' research was aimed at producing amyloid-based materials from an accessible source of protein. To do this, research was directed to the factors that influenced amyloid formation. Reliable protocols were established for the production of amyloid in a model system and then the protocols were experimented with using a protein source suitable for use in an industrial production scale.

Wheat Protein Classification

Wheat flour proteins have conventionally been characterised on the basis of solubility (Osborne 1907), into the following groups: albumins and globulins (soluble in dilute salt), gliadins (soluble in aqueous ethanol), and glutenins (soluble in dilute acid). These groups are outlined in Table 1. The glutenins can be classified further into high molecular weight (HMW) and low molecular weight (LMW) categories. The albumins and globulins represent the non-gluten fraction of wheat protein, and the gliadins and glutenins the gluten fraction.

TABLE 1
The traditional classification of wheat proteins, based on sohibility.
Adapted from Frazier (1992)

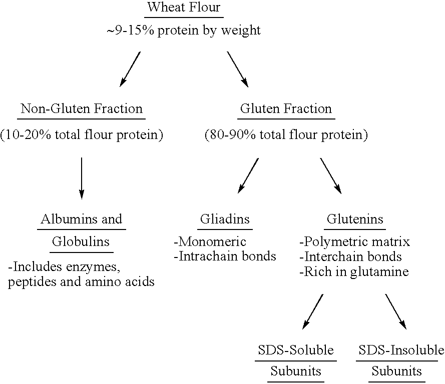

The ability to isolate wheat protein into fractions of varying levels of purity allowed the authors to investigate the influence of protein purity in the wheat protein system, from heterogeneous mixtures of glutenins right down to solutions containing a glutenin subunit encoded by a single gene locus. For this initial research. the glutenin fraction was separated into protein components that were soluble in SDS-phosphate buffer, and protein components that were not, using the extraction methods described by Gerrard et al. (2001), based on the protocols of Osborne (1907). The protein groups that did not solubilise after the initial buffer treatment were sonicated briefly to increase their solubility. The addition of a reducing agent was necessary to aid the separation of the HMW- and LMW-glutenin subunits (Egorov et al. 1998). The isolated Glu-1A gene locus encoded HMW-glutenin subunit 1 and Glu-1D gene locus encoded HMW-glutenin subunit pair 5+10 were supplied by K. Sutton, Crop and Food Research, and sourced from commercial wheat varieties using the methods outlined by Sutton (Sutton 1991).

Attempts were made to induce the formation of amyloid from protein fractions extracted from wheat flour. It is well known to those practised in the art that the fractionation protocols presented herein represent the most commonly used procedures for wheat proteins and that alternative fractionation schemes (e.g. use of iso-propanol or acetic acid) will also yield similar heterogeneous protein mixtures that could be used as starting materials for amyloid formation.

1. Materials

Unless otherwise stated, all chemicals and reagents were purchased from Sigma Chemical Company Ltd., Aldrich Chemicals or BDH Laboratory Supplies, and were of analytical grade.

The wheat derived protein used in the described extractions was either purchased from a local supermarket, or milled and supplied by Crop and Food Research. The flour sources were of varying levels of quality and composition.

The HMW glutenin subunits were extracted and alkylated using the method of Sutton (1991). The A-gliadin fraction was supplied by Crop and Food Research, and extracted using in-house techniques.

pH measurements were obtained using an UltraBasic UB10 pH meter purchased from Denver Instrument Co. and fitted with a high-performance glass body pH/ATC electrode. The electrode was calibrated daily against standard buffers at pH 4.0, 7.0 and 9.0, purchased from BDH Laboratory Supplies.

Polyacrylamide gel electrophoresis was routinely run using a Bio-Rad Mini PROTEAN® Cell kit powered by a Bio-Rad 300 Power pack, and Gradipore precast 8-16% acrylamide gradient iGels.

[image]

Ultraviolet (UV) spectroscopy was performed using a Hewlett Packard 8452A Diode Array Spectrophotometer interfaced with a personal computer running Hewlett Packard 8452A UV-visible operating software.

Fluorescence Spectroscopy was performed using a Cary Eclipse Varian Fluorescence Spectrophotometer interfaced with Cary Eclipse version 2 operating software.

Microscopy imaging was carried out using a J. Swift & Son optical microscope fitted with a cross-polarising lens.

Electron micrographs were obtained using a JOEL JEM-1200 EX transmission electron microscope operating at 80 kV.

2. Wheat Protein Extraction

Wheat flour was separated into four protein fractions using the methods of Gerrard et al. (2001), based on the protocols of Osborne (Osborne 1907). This sequential four step extraction procedure was carried out on a scale of between 100 mg to 5 g of wheat flour. Firstly, albumins and globulins were extracted using a 2% (w/v) sodium chloride solution (0.4 mL per 100 mg of wheat flour). The mixture was pulse vortex mixed every 5 min for 30 min, then centrifuged at 10,000 g for 5 min. The clarified supernatant containing albumins and globulins was stored at −20° C. The resultant pellet was resuspended in 70% (v/v) aqueous ethanol solution (0.4 mL per 100 mg of initial wheat flour weight) to extract the gliadin protein fraction. This mixture was pulse vortex mixed every 5 min for 30 min, then centrifuged at 10,000 g for 5 min. The clarified supernatant containing gliadins was stored at −20° C. The solid fraction was re-extracted in SDS-buffer (0.5% (w/v) SDS, 0.05 M phosphate, pH 6.9, 0.4 mL per 100 mg of initial wheat flour weight) to separate the SDS-soluble glutenin protein fraction. The mixture was pulse vortex mixed every 5 min for 30 min, then centrifuged at 10,000 g for 5 min. The resultant clarified supernatant was stored at −20° C. Finally, the residual pellet was extracted with a SDS-buffer (0.5% (w/v) SDS, 0.05 M phosphate, pH 6.9, 0.4 mL per 100 mg of initial wheat flour weight). The heterogeneous mixture was sonicated at. 30 W for 15 sec using a Branston model 250 sonic disruptor. After vortexing, the samples were centrifuged at 10,000 g for 5 min. The clarified SDS-insoluble glutenin supernatant was stored at −20° C. Each protein fraction was freeze dried and restored at −20° C.

3. HMW-Glutenin Extraction and Alkylation Procedure

Wheat flour samples were extracted on a 100 mg scale. The total glutenin fraction of the wheat flour was extracted using a propan-1-ol/dithiothreitol (DTT) mixture (0.625 mL of 50% (v/v) propan-1-ol, 1% (w/v) DTT) heated at 60° C. for 30 min with pulse vortex every 5 min. The samples were centrifuged at 20,000 g for 10 min. An aliquot (0.5 mL) of the supernatant was used in the subsequent procedure. The concentration of propan-1-ol was then adjusted to 60% (v/v). The mixture was pulse vortexed and allowed to stand at room temperature for 30 min. The precipitated HMW-glutenin subunits were isolated by centrifuging at 3000 g for 5 min and were washed three times with 60% (v/v) propan-1-ol (1 mL). The HMW-glutenins were dissolved in reducing buffer (0.5 mL, 0.08M Tris-HCl, pH 7.5, 50% propan-1-ol (v/v) 1% dithiothreitol (DTT), (w/v)) heated at 60° C. for 30 min, with agitation every 10 min. The resulting reaction mixture was allowed to cool to room temperature before addition of the alkylating buffer (0.5 mL, 0.08 M Tris-HCl, pH 7.5, 50% propan-1-ol (v/v) 3% 4-vinylpyridine (v/v)). Care was required with this step as the 4-vinylpyridine causes severe burns. The alkylation reaction mixture was heated in a water bath at 60° C. for 15 min. After cooling to room temperature, this solution was acidified by the addition of glacial acetic acid (0.1 mL). Salts were removed by dialysing in 0.01 M acetic acid solution overnight. Finally the solvent was removed by freeze drying to yield alkyated HMW-glutenin subunits that were soluble in weak acids.

4. Attempt to form Wheat Flour Protein Fibrils

All extracted wheat flour protein fractions were resuspended in solutions at the concentrations given in the table below (Table 2).

TABLE 2
The solutions used for the wheat fraction incubations.

| | | Approx. |
|---|---|---|
| | | Concentration |
| Protein Fraction | Resuspending Solution | (mg/mL) |
| Gliadins | 70% aqueous ethanol (v/v) | 2 |
| Gliadins | 70% aqueous ethanol (v/v) | 2-10 |
| SDS-soluble | 0.5% SDS (w/v), 0.05 M | 2-20 |
| Glutenins | phosphate, pH 6.9 | |
| SDS-insoluble g | 0.5% SDS (w/v), 0.05 M | 2-20 |
| Glutenins | phosphate, pH 6.9 or, | |
| | Nanopure Water | |
| SDS-insoluble | Nanopure Water | 2-10 |
| Glutenins | | |
| HMW-Glutenins | Dilute Acid (<0.5% HCl (v/v) | 10 |
| HMW-Glutenin | Dilute Acid (<0.5% HCl (v/v) | 10 |
| Subunit 1 (Glu-1A) | | |
| HMW-Glutenin | Dilute Acid (<0.5% HCl (v/v) | 10 |
| Subunit pair | | |
| 5 + 10 (Glu-1D) | | |

These different reaction mixtures were subjected to a wide range of experimental conditions, including:
temperatures of 25° C.-37° C.
addition of acids (H₂SO₄, HCl, various concentrations including 0.5 M)
addition of organic solvents (30% trifluoroethanol, 80% ethanol)
addition of reducing compounds (mercaptoethanol, various concentrations including 1 M)
addition of denaturants (urea, various concentrations including 2 M)
addition of preformed insulin ‘seeding’ molecules (10-20 μL of insulin amyloid, prepared fresh using the flash-freeze protocol of Klunk et al. (1989b)).

The appropriate reagents were added to the reaction mixtures before incubation. All treatments were prepared in triplicate. For each treatment system samples frozen without heating were used as controls. Aliquots (120 μL) were removed at approximately 10 day intervals for a period of up to 70 days. These aliquots were frozen at −20° C. until required for analysis by a combination of ThT assay, CR assay and TEM imaging.

5. Protein Analysis

5.1 Protein Concentration

Protein concentration in the resuspended solutions was determined using a modified version of the Bradford method (1976). A standard curve with bovine serum albumin (BSA) or insulin was determined daily. The absorbance is linear for concentrations from 1 to 15 μg/mL of BSA.

The protein concentration of unknown solutions was determined by the addition of Bio-Rad Bradford reagent (200 μL) to solution (800 μL). If the absorbance reading was outside the linear range of the standard curve it was necessary to dilute (or concentrate) the sample to ensure accuracy of the results. After mixing and incubation at room temperature for 6 min, the absorbance of each sample was measured at 595 mm against a distilled water blank. All analyses were performed in triplicate.

5.2 SDS-PAGE Electrophoresis

Each sample was placed in a labelled Eppendorf tube and resusupended in a loading buffer (0.5 M Tris-HCl Buffer pH 6.8, 5% SDS (w/v), 20% glycerol (v/v),10% 2-β-mercaptoethanol (v/v), 0.5% bromophenol (w/v)). The samples were placed in a boiling water bath for 2 min to help solubilise the samples and to aid the reduction of disulfide bonds. Protein samples were run against wide range molecular weight markers purchased from Sigma (6,500-205,000 Da). Each boiled protein sample and marker was loaded into separate wells in the polyacrylamide gel. Samples were electrophoresed at a constant voltage of 150 V in running buffer (20 mM Tris, 20 mM glycine, 0.5% SDS w/v pH 8.3) at 4° C. The proteins in the resultant polyacrylamide gel were stained in a solution of Coomassie brilliant blue stain (0.1% Coomassie brilliant blue (10% v/v), glacial acetic acid (50% v/v), methanol, dH₂O (40% v/v), pre-filtered at room temperature). After two hours of staining the gel was transferred into a destaining solution (glacial acetic acid (10% v/v), methanol (5% v/v), dH₂O (85% v/v)) at room temperature.

6. Detection of Amyloid

The protein samples were analysed using a range of analytical techniques developed specifically for the detection of amyloid material. Samples could be stained using the dye Congo red (Westermark et al. 1999), or via the use of calorimetric (Klunk et al. 1989a, Klunk et al. 1989b) or fluorescence (LeVine 1999) assays.

6.1 Congo Red Stain

Samples were stained for amyloid following the procedure of Westermark (1999). The protein samples were fixed onto glass slides pre-coated in poly-L-lysine, prepared by submerging in a solution of 0.1% poly-L-lysine overnight, with drying at 337° C. To stain the samples, the slides were soaked in prefiltered working solution A (170 mM NaCl, 80% ethanol (v/v), NaOH (0.001%, added prior to filtering)) for 20 min. Slides were then transferred directly to a prefiltered working solution B (0.2% Congo Red (w/v), 170 mM NaCl, 80% ethanol (v/v), NaOH (0.001%, added prior to filtering)) and soaked for a further 20 min. This was followed by two brief washes in 100% ethanol.

Using this method, samples appeared as red-orange material under a light microscope. When viewed under cross-polarised light, where one light filter is rotated at 90° with respect to a second light filter, a bright green or ‘apple green’ birefingence could be observed in areas where amyloid was present.

6.2 Congo Red Assay

The method for this binding assay involves a modification of the protocol described by Klunk et al. (1989). The spectral measurements were taken using a HP-8452A diode array spectrophotometer set in the wavelength-scanning mode to read from 300 to 700 nm.

To analyse the wheat protein fractions, a solution of Congo red (1 mM) was prepared in a HEPES buffer (0.01 M HEPES, 2.7 mM KCl, 0.137 M NaCl, pH 7.4), containing 10% ethanol to prevent the formation of micelles (micro-aggregation). This solution was filtered three times through Gelman extra-thick glass fibre filters before use, and stored at room temperature protected from light. The protein concentration of the samples to be tested was between 10-20 μM. All samples were assayed in duplicate. Congo red solution was diluted with phosphate buffer immediately prior to use in the spectrophotometric assays, typically to give a final Congo red concentration of 10 μM in the protein sample solution to be analysed. Congo red solution and protein solution were mixed in 1 mL cuvette and allowed to stand at room temperature for 30 min before spectral analysis. The spectrum of the resulting mixture was measured between 300-700 nm. The spectra of buffer, buffer with Congo red and buffer with protein were the experimental controls. A shift of the spectrum to longer wavelengths and a new point of maximal difference at 540 nm is evidence for the presence of amyloid (Klunk et al. 1999).

6.3 Thioflavin T Fluorescence Assay

The Thioflavin T (ThT) method used throughout this research was based on the protocols of Le Vine (11999).

General Procedure

The wavelength of excitation was 450 nm. The complete emission spectra were recorded, over a range that included the wavelength of 482 mm. Both excitation and emission slits were set to 5 nm.

In the cuvette, protein sample was added to a Tris buffer (50 mM Tris, 100 mM NaCl, pH 7.5) containing ThT (5 to 10 μM). The final concentration of protein in the cuvette was typically 20 μg/mL. The resulting solution was mixed and left to stand at room temperature for 3 min to allow binding between the dye and protein to equilibrate, before the emission spectra were recorded.

The spectra of buffer only, buffer with ThT and buffer with protein were the experimental controls. Emission spectra for all samples were assayed in duplicate.

The experiments were also conducted in well plates that were standardised as for the cuvette procedure. The wheat protein concentration for these assays was approximately 0.5 mg/mL. The final volume in the well plates was 200 μL.

6.4 Transmission Electron Microscopy Imaging

The protein samples were analysed by TEM based on the protocols described by Whittingham et al. (2002) and Burke and Rougvie (1972). TEM samples were prepared by placing small aliquots (5 mL) of protein fibril samples on carbon-coated copper grids. Grids were then rinsed with distilled water, negatively stained with 1% uranyl acetate, and dried before imaging commenced.

Specific Procedure for the Production of Amyloid Fibrils

The general protocols used can be found above. Additional specific details are described below:

### EXAMPLE 1

The wheat flour was separated into fractions using the protocols outlined in Methods 2 (Wheat Protein Extraction) and 3 (HMW-Glutenin Extraction and Alkylation Procedure), using initially both flour purchased from the supermarket (extractions for gliadin and glutenin fractions) and flour supplied by Crop and Food Research (as above but also for HMW-glutenin subunits). Flours from specific wheat cultivars were used to extract the HMW-glutenins but these did not appear to form fibrils (Glu-1A gene locus encoded subunit 1, and Glu1D gene locus encoded subunit pair 5+10).

### EXAMPLE 2

The supernatants containing the solubilised protein fractions were pooled and lyophilized to concentrate the protein content. Incubations were carried out involving protein fractions that had not been lyophilised and hence were less concentrated (i.e. <<10 mg/ml). These samples did not conclusively form fibrils. However, based on the successful results with the higher concentration (lyophilised) samples, the possibility that these samples could have (given more time) can not be excluded.

### EXAMPLE 3

The lyophilised protein fractions were then redissolved in either the appropriate extraction buffer or nanopure water (Table 2), typically to give a final protein concentration of approximately 10 mg/ml. The protein samples tested ranged in final concentration from 6.5-15 mg/mL, but typically were 10 mg/mL.

### EXAMPLE 4

The majority of the protein samples were then used for the various incubation treatments with no further treatment, while a selection of the samples were dialysed against distilled water to remove SDS and phosphate buffer salts.

### EXAMPLE 5

The samples were then placed in 1.5 mL Eppendorf tubes and incubated at either 25° C. or 37° C. The volume of each tube was initially between 1-1.5 mL, with each sample condition prepared in triplicate. A separate volume of at least 120 μL was frozen at −20° C. (for glutenins) or −80° C. (for gliadins, in the ethanol based solution) as a time zero control.

TABLE 3
The range of incubation conditions investigated. The positive and negative signs refer
to the samples that tested positive or negative for amyloid content respectively.

| Treatment | | | Protein Fraction | | | | |
|---|---|---|---|---|---|---|---|
| | Approx. | | | SDS- | SDS- | | HMW- |
| Temp | Conc. | | | Insoluble | Insoluble | HMW- | Glutenin |
| (° C.) | (mg/ml) | Conditions | Gliadin^{a} | Glutenin^{b} | Glutenin^{c} | Glutenin^{d} | Subunits^{e} |
| −20 | 2 | pH 2 | − | − | − | | |
| −20 | 2 | pH 3 | − | − | − | | |
| −20 | 2 | pH 6-7 | − | − | − | | |
| −20 | 10 | pH 6-7 | − | − | − | − | − |
| 25 | 10 | pH 6-7 | − | + | +/+ | − | − |
| 25 | <10 | pH 6-7, dialysed | | − | − | | |
| 25 | 10 | pH 6-7, 20 μL insulin seed^{g} | − | + | −/− | | |
| 25 | 10 | pH 6-7, 0.5M H₂SO₄ | − | + | +/− | | |
| 25 | 10 | pH 6-7, 0.5M HCl | − | + | −/− | | |
| 25 | 10 | pH 6-7, 2M urea | − | − | −/− | − | − |
| 25 | 10 | pH 6-7, 30% (v/v) TFE | − | − | −/− | | |
| 25 | <20 | pH 6-7, dialysed | − | − | − | | |
| 37 | 2 | pH 6-7 | − | − | − | | |
| 37 | 2 | pH 2, 30% TFE | − | − | − | | |
| 37 | 2 | pH 2, boil 90 sec, incubate | − | − | − | | |
| 37 | 2 | pH 3, boil 90 sec. incubate | − | − | − | | |
| 37 | 2 | pH 2, 0.5M EDTA | − | − | − | | |
| 37 | 2 | pH 3, 0.5M EDTA | − | − | − | | |
| 37 | 2 | pH 2, 2M urea | − | − | − | | |
| 37 | 2 | pH 3, 2M urea | − | − | − | | |
| 37 | 2 | pH 2, 10 μl insulin seed^{g} | − | − | + | | |
| 37 | 2 | pH 3. 10 μl insulin seed^{g} | − | + | + | | |
| 37 | 10 | pH 6-7, | − | + | +/+ | | |
| 37 | 10 | pH 6-7, 20 μL insulin seed | − | + | +/− | | |
| 37 | 10 | pH 6-7, 0.5M H₂SO₄ | − | − | −/− | | |
| 37 | 10 | pH 6-7, 0.5M HCl | − | − | +/− | | |
| 37 | 10 | pH 6-7, 2M urea | − | + | +/+ | | |
| 37 | 10 | pH 6-7, 4M urea | | − | | | |
| 37 | 10 | pH 6-7, 4M urea, 30 sec sonication | | − | | | |
| 37 | 10 | pH 6-7, 6M urea | | + | | | |
| 37 | 10 | pH 6-7, 30% (v/v) TFE | − | − | +/+ | | |

^{a}70% (v/v) aqEtOH or 10% acetic acid.
^{b}0.5% SDS (w/v), 0.05M phosphate. pH 6.9.
^{c}0.5% SDS (w/v), 0.05M phosphate, pH 6.9.
^{d}Nanopure water.
^{e}Glu-1A gene locus encoded HMW-glutenin subunit 1 and Glu-1D gene locus encoded HMW-glulenin subunit pair 5 + 10.
^{f}After lyophilisation, the insoluble glutenin fractions were solubilised either in the original extraction buffer or in nanopure water. The first result refers to the SDS-phosphate solutions, the second the nanopure water solutions.
^{g}Insulin seed induced to form amyloid after incubation in a pH 2 HCl-dH₂O solution, 50° C., 48 hr.

### INDUSTRIAL APPLICABILITY

The invention provides a method for the formation of amyloid that can be used as a model to study amyloid assembly and structure and particularly to study the formation of amyloid in diseases such as Alzheimer's, Parkinson's and encephalopathies with a view to understanding how these diseases could be treated. The invention also provides a novel new source of amyloid protein, suitable for applications: in nanotechnology (e.g. generation of silver or other metal nanowires); as material scaffolds for tissue and bone growth; as target-selective porous channels in films and membranes (e.g. for bioremediation); for strength enhancement of composites and films; and as biosensors.

### REFERENCES

Bellotti. V. P. Mangione, and M. Stoppini. 1999. Biological activity and pathological implications of misfolded proteins. Cellular and Molecular Life Sciences 55: 977-991.
Bradford, M. A. 1976. A rapid and sensitive method for the quantitation of microgram quantities of protein utilising the prinicle of protein dye binding. Analytical Biochemistry. 72: 248-254.
Burke, M. J., and M. A. Rougvie. 1972. Cross-ÿ protein structures. 1. Insulin fibrils. Biochemistry 11: 2435-2439.
Chen. S., V. Berthelier, J. B. Hamilton, B. O'Nuallain, and R. Wetzel. 2002. Amyloid-like features of polyglutamine aggregates and their assembly kinetics. Biochemistry 41: 7391-7399.
Chiti, F., P. Webster, N. Taddei, A. Clark, M. Stefani, G. Ramponi, and C. M. Dobson. 1999. Designing conditions for in vitro formation of amyloid protofilaments and fibrils. Proceedings of the National Academy of Sciences of the United States of America 96: 3590-3594.
Dobson, C. M. 2001. The structural basis of protein folding and its links with human disease. Philosophical Transactions of the Royal Society of London. Series B, Biological Sciences 356: 133-145.
Dowd, C., and F. Bekes. 2002. Large-scale expression and purification of high-molecular-weight glutenin subunits. Protein Expression and Purification 25: 97-104.
Egorov, T. A., T. I. Odintsova, P. R. Shewry, and A. S. Tatham. 1998. Characterisation of high Mᵣ wheat glutenin polymers by agarose gel electrophoresis and dynamic light scattering. FEBS Letters 434: 215-217.
Gerrard, J. A., S. E. Fayle, P. A. Brown, K. H. Sutton, L. Simmons, and I. Rasiah. 2001. Effects of microbial transglutaminase on the wheat proteins of bread and croissant dough. Journal of Food Science 66: 782-786.
Graveland, A., M. H. Henderson, M. Pagues, and P. A. Zandbelt. 1995. Composition and structure of gluten properties. Pages 90-98 in J. D. Schofield, ed. Wheat Structure, Biochemistry, and Functionality. The Royal Society of Chemistry, Reading. U.K.
Guijarro, J. I., M. Sunde, J. A. Jones, I. D. Campbell, and C. M. Dobson. 1998. Amyloid fibril formation by an SH3 domain. Proceedings of the National Academy of Sciences of the United States of America 95: 4224-4228.
Klunk, W. E., J. W. Pettegrew, and D. J. Abraham. 1989a. Quantitative evaluation of Congo red binding to amyloid-like proteins with a ÿ-pleated sheet conformation. The Journal of Histochemistry and Cytochemistry: Official Journal of the Histochemistry Society 37: 1273-1281.
Klunk, W. E. 1989b. Two simple methods for quantifying low-affinity dye-substrate binding. The Journal of Histochemistry and Cytochemistry: Official Journal of the Histochemistry Society 37: 1293-1297.
LeVine, H., III. 1999. Quantification of ÿ-sheet amyloid fibril structures with thioflavin T. Methods in Enzymology 309: 274-284.
Maloy, A. L., D. S. Longnecker, and E. R. Greenberg. 1981. The relation of islet amyloid to the clinical type of diabetes. Human Pathology 12: 917-22.
Nielsen, L., R. Khurana, A. Coats, S. Frokjaer, J. Brange, S. Vyas, V. N. Uversky, and A. L. Fink. 2001. Effect of environmental factors on the kinetics of insulin fibril formation: Elucidation of the molecular mechanism. Biochemistry 40: 6036-6046.
Osborne, T. B. 1907. The proteins of wheat kernal. Publications of the Carnegie Institution No 84. Judd and Detweiler, Washington, USA.
Sutton, K. H. 1991. Qualitative and quantitative variation among high molecular weight subunits of glutenin detected by reversed-phase high-performance liquid chromatography. Journal of Cereal Science. 14: 25-34.
Takahashi, Y., A. Ueno, and H. Mihara. 1998. Design of a peptide undergoing ÿ-ÿ structural transition and amyloid fibrillogenesis by the introduction of a hydrophobic defect. Chemistry—A European Journal 4: 2475-2484.
Westermark, G. T., K. H. Johnson, and P. Westermark. 1999. Staining methods for identification of amyloid in tissue. Methods in Enzymology 309: 3-25.
Whittingham, J. L., D. J. Scott, K. Chance, A. Wilson, J. Finch, J. Brange, and G. G. Dodson. 2002. Insulin at pH 2: Structural analysis of the conditions promoting insulin fibre formation. Journal of Molecular Biology 318: 479-490.
Wrigley, C. W. 1996. Giant proteins with wheat power. Nature 381: 738-739.
Zanuy, D., B. Ma, and R. Nussinov. 2003. Short peptide amyloid organization: Stabilities and conformations of the islet amyloid peptide NFGAIL. Biophysical Journal 84: 1884-1894.

1. A method of amyloid fibril formation using a heterogeneous source of protein as a starting material.

2. A method according to claim 1 wherein the protein source has a high glutamine content.

3. A method according to claim 1 wherein the protein source is from wheat source or a wheat protein sequence from a microbiological expression system.

4. A biomaterial containing amyloid fibrils made according to claim 1.

5. A method of producing amyloid fibrils from a high molecular weight, heterogeneous source of protein as a starting material.

6. A method according to claim 5 wherein the starting material is a wheat protein.

7. A method according to claim 6 in which the wheat protein is from a microbiological expression system.

8. A method according to claim 6 wherein the starting material is an SDS-soluble wheat protein fraction.

9. A method according to claim 7 wherein the starting material is an SDS-insoluble wheat protein fraction.

10. Amyloid fibrils produced by the method of claim 1.

11. A method of producing amyloid fibrils derived from wheat, comprising: (a) Providing wheat protein, crudely fractionated from a milled flour; (b) Separating a heterogeneous protein mixture on the basis of solubility; (c) Obtaining protein solutions containing a broad range of proteins of varying molecular weights and compositions;
(d) Incubation of these fractions at moderate temperatures, typically in the presence of specific compounds known to destabilise a protein's structure to induce the formation of amyloid

12. Amyloid fibrils produced by the method of claim 11.

13. A method according to claim 1 wherein the method is performed in vitro.

14. A method according to claim 1 wherein a denaturing compound is added to induce the formation of amyloid-like structures.

15. A method according to claim 14 wherein the denaturing compound is one or more of urea, a thiol containing reductant (e.g. dithiothreitol (DTT)), oran acid (e.g. H2SO4,HCl).

16. A method according to claim 15 wherein the pH range is 2-7.5, preferably 5-7.5.

17. A method according to claim 15 wherein the temperature range is 20-70 C, preferably about 50 C.

18. A method according to claim 14 wherein the denaturing compound is incubated with the protein source at 25 C for up to 105 days.

19. A method according to claim 14 wherein the denaturing compound is incubated with the protein source at 37° c. for up to 105 days.

20. A method according to claim 18 wherein the incubated protein is a wheat protein.

21. A method according to claim 20 wherein the incubated protein is substantially a wheat protein sequence from a microbiological expression system.

22. A method according to claim 1 wherein an extraneous amyloid fibril is added as a seed to induce amyloid fibril formation in the wheat protein treatments.

23. Amyloid fibrils produced by the method of claim 13.

24. The method of claim 22 wherein the amyloid fibril comprises insulin fibril.
